(19) United States
(12) Patent Application Publication
(10) Pub. No.: US 2022/0387617 A1
(43) Pub. Date: Dec. 8, 2022

(54) USE OF CD200 PROTEIN AND CD200 FUSION PROTEIN IN PREPARING A DRUG FOR TREATING PSORIASIS

(71) Applicant: CHINA PHARMACEUTICAL UNIVERSITY, Nanjing (CN)

(72) Inventors: Hanmei XU, Nanjing (CN); Dongping LI, Nanjing (CN); Xinrong JIN, Nanjing (CN)

(21) Appl. No.: 17/761,538

(22) PCT Filed: Oct. 26, 2020
(86) PCT No.: PCT/CN2020/123629

§ 371 Date: Mar. 17, 2022

(30) Foreign Application Priority Data
Sep. 23, 2019 (CN) 201910898038.8

(51) Int. Cl. A61K 47/68; A61K 38/17; A61P 17/06

(52) U.S. Cl. CPC A61K 47/6849; A61K 38/1774; A61P 17/06

(57) ABSTRACT

A use of a CD200 extracellular domain protein or a fusion protein formed from a CD200 extracellular domain protein and an Fc fragment in preparing a drug for treating psoriasis is disclosed.

### TECHNICAL FIELD

The present invention relates to the field of biopharmaceuticals, and in particular to use of CD200 protein and CD200 fusion protein in preparation of drugs for treating psoriasis.

### BACKGROUND

Psoriasis is a common chronic and inflammatory skin disease with characteristic skin injury and prone to recurrence. The disease features high morbidity, chronicity, stubbornness, and proneness to recurrence after healing, which causes great physical pain and great mental stress on a patient. As a common chronic autoimmune disease, psoriasis is caused by the interaction between keratinocytes and immune cells, which is related to inflammatory skin and affects of the world's population. Symptoms of psoriasis include erythema, skin hyperplasia, scales, and keratinocyte excessive proliferation, and lesions include acanthosis nigricans caused by keratinocyte excessive proliferation and lymphadenopathy and parakeratosis caused by abnormal keratinocyte differentiation.

Although both psoriasis and systemic lupus erythematosus are autoimmune diseases, their causes, treatment means, and treatment drugs are different. In addition, psoriasis and inflammatory skin diseases are also different in the causes, treatment means, and treatment drugs.

Systemic lupus erythematosus (SLE) is an autoimmune inflammatory connective tissue disease that affects multiple organs and occurs in young women. In some severe cases, the conditions can sometimes relieve themselves. Some patients have a “transient” attack, and the disease may disappear completely after a short course of several months. At present, the cause of systemic lupus erythematosus has not been confirmed. A large number of studies have shown that heredity, endocrine, infection, immune abnormality and some environmental factors are related to the disease. Under the interaction of genetic factors, environmental factors, estrogen levels and other factors, T lymphocytes decrease, functions of T suppressor cells decrease, B cells proliferate excessively, and a large number of autoantibodies are produced, and are binded with corresponding autoantigens in the body to form corresponding immune complexes, which are deposited in skin, joints, small blood vessels, glomerulus and other parts. With the participation of a complement, acute and chronic inflammation and tissue necrosis are caused, or antibodies directly interact with histocyte antigens, causing cell destruction (for example, specific antigens of red blood cells, lymphocytes and platelet walls bind to corresponding autoantibodies, causing hemolytic anemia, lymphopenia and thrombocytopenia respectively), thereby leading to multi-system damage of the body. Clinical treatment means of systemic lupus erythematosus is intravenous drip or oral administration, and common drugs are non-steroidal anti-inflammatory chugs, antimalarials, glucocorticoids, immunosuppressants and the like.

The pathogenesis of psoriasis may involve multiple aspects, including genetics, infection, immune abnormalities, endocrine and other factors. A considerable number of patients have a family history of disease, and some families have obvious genetic predispositions. It is generally believed that people with a family history account for about 30%. The morbidity varies greatly among different races. At present, it has been confirmed that streptococcal infection is related to the onset and prolonged course of psoriasis from the aspects of humoral immunity, cellular immunity, bacterial culture, treatment, and the like. In patients with psoriasis, Staphylococcus aureus infection can make skin lesions worse, which is related to the superantigen of Staphylococcus aureus exotoxin. A large number of studies have proved that psoriasis is an immune-mediated inflammatory skin disease, in which inflammatory macrophages play a key role, and its pathogenesis is related to inflammatory cell infiltration and inflammatory factors. Some female patients have reduced or even disappeared skin lesions after pregnancy, and aggravated skin lesions after delivery. The clinical treatment means of psoriasis are external and oral administration. Common drugs for external administration include vitamin D3 analogs, glucocorticoids, tretinoin, tars, immunosuppressants, and the like. Common oral drugs are methotrexate, tretinoin, antibiotics, and the like. Glucocorticoids play a key role in the treatment of systemic lupus erythematosus. However, oral glucocorticoids can also be used to treat psoriasis. This type of drug should not be used for psoriasis in a conventional system because the effect is not obvious. After drug withdrawal, symptoms are worse, or even acute pustular psoriasis or erythrodermic psoriasis may be induced.

Dermatitis is a general term referring to inflammatory diseases of skin caused by various internal and external infections or non-infectious factors. Dermatitis, which is not an independent disease, has complex and diverse causes and clinical manifestations, and is recurrent, making clinical treatment more difficult. The causes of dermatitis and eczema are very complicated, and may be related to the following factors. Internal factors: chronic infection focus (such as chronic cholecystitis, tonsillitis, and intestinal parasitic diseases), endocrine and metabolic changes. (such as menstrual disorder and pregnancy), blood circulation disorder (such as varicose veins of calf), neuropsychiatric factors genetic factors and the like. External factors: The disease can be induced or aggravated by food (such as fish, shrimp, beef, and mutton), inhalants (such as pollen and dust mites), living environment (such as cold, hot, and dryness), animal fair, and various physical and chemical substances (such as cosmetics, soap, and synthetic fibers). Clinically, drugs may be administrated externally, orally or intravenously. For external administration, glucocorticoid cream can be used in an acute phase when the exudation is less, 3% boric acid solution can be used for cold and wet compress when the exudation is much, and after the exudation is reduced, glucocorticoid cream is used or is used alternately with an oil agent. Glucocorticoid emulsion and paste may be used in a subacute stage, and antibiotics can be added to prevent secondary infection. In the chronic stage, an ointment, a plaster or a film coating agent is selected. Intractable localized skin lesions may be treated by intradermal injection of glucocorticoid. Acute stage and subacute stage: Intravenous injection of calcium, vitamin C, and the like or procaine vein occlusion can be used; \textcircled{1} For patients with a skin lesion area less than 30%, externally used drugs may be combined with antihistamines and compound glycyrrhizin. \textcircled{2} For patients with a skin lesion area greater than or equal to 30%, 10% calcium gluconate, sodium thiosulfate or a compound glycyrrhizin preparation may be used intravenously. (2) Chronic stage: \textcircled{1} For patients with a skin lesion area, less than 30%, externally used drugs may be used by combining with antihistamines and compound glycyrrhizin and the like taken orally; for patients with a poor curative effect, a Tripterygium wilfordii preparation or an immunosuppressant may be added for a short time, and the drugs are withdrawn after the condition is controlled. \textcircled{2} Most patients with a skin lesion area greater than 30% need to orally take compound glycyrrhizin, a Tripterygium wilfordii preparation or immunosuppressant, an immunomodulator and an antihistamine.

In conclusion, it can be learned that systemic lupus erythematosus and inflammatory skin diseases are different from psoriasis in pathogenesis, therapeutic drugs, treatment means and therapeutic effects.

CD200 and CD200 receptors (CD200R) are highly conserved type I transmembrane cell surface glycoproteins belonging to the immunoglobulin superfamily (IgSF). CD200 may be expressed in a variety of cells, including, T cells, B cells, dendritic cells, and neuron cells. A CD200 molecule is composed of three domains: all extracellular domain, a transmembrane domain and an intracellular domain, and its intracellular structure lacks a signal motif CD200R is a highly conserved glycosylated protein with a molecular weight ranging from 60 kDa, to 110 kDa, which depends on the degree of glycosylation and a type of expression cells. CD200R is mainly expressed by bone marrow cells such as macrophages and microglia. There are five types of CD200R, namely CD200R1 to CD200R5, where CD200R1 has the highest binding affinity for CD200. The signal pathway of CD200/CD200R1 involves inhibiting degranulation of mast cells and basophils, down-regulating of macrophage functions, and the like. CD200/CD200R1 signal transduction is closely related to the prevention of autoimmune diseases, but the role of CD200 CD200R1 signal transduction in the pathogenesis of psoriasis is still unknown.

It is shown that in use of a CD200 antibody (CN103690947A), use of a CD200 mutant (CN109219614A), use of a CD200R antibody (CN101679519A) use of CD200 in treating systemic lupus erythematosus (CN102698266A) an anti-CD200 antibody therapy method (CN102918062A), regulation of bone mass through osteoclast differentiation by using CD200 and CD200R (CN101687033), and a CD200 blocker and a usage method (JP2018537433) at home and abroad, no study is conducted on treatment of psoriasis by using CD200, and CD200 may play a role in the pathogenesis of inflammatory skin diseases (Akman-Karakas A et al., (2013) Med Sci Monit. 19:888-91), but the pathogenesis of psoriasis has not been studied.

### SUMMARY

The Sequence Listing created on Mar. 16, 2022 with a file size of 24 KB, and filed herewith in ASCII text file format as the file entitled “Sequenee_Listing_G205RAYT0001US.TXT,” is hereby incorporated by reference in its entirety.

### 1. To-be-Resolved Problem

To farther study a mechanism of action of CD200 protein and CD200 fusion protein, further research is performed in the present invention, and it has been found that CD200 protein and CD200 fusion protein can play an important role in the treatment of psoriasis.

### 2. Technical Solutions

To achieve the foregoing objective, the technical solution adopted in the present invention is as follows:

Use of a CD200 extracellular domain protein or a fusion protein formed by a CD200 extracellular domain protein and an Fc fragment in preparation of drugs for treating psoriasis.

A nucleotide sequence of the CD200 extracellular domain protein is shown in SEQ ID NO: 1.

An amino acid sequence of the CD200 extracellular domain protein is shown in SEQ ID NO: 2.

Fc is an Fc fragment of human IgG1, IgG2 or IgG3, and a nucleotide sequence of the Fc fragment of IgG1, IgG2 or IgG3 is shown in SEQ ID NO: 3, SEQ ID NO: 4 or SEQ ID NO: 5 respectively.

An amino acid sequence of the Fc fragment of IgG1, IgG2 or IgG3 is shown in SEQ ID NO: 6, SEQ ID NO: 7 or SEQ ID NO: 8 respectively.

A complex is provided, where the complex is obtained by adding one or more pharmaceutically acceptable excipients to the fusion protein according to claim 1.

The excipients include a diluent, a filler, an adhesive, a wetting agent, an absorption enhancer, a surfactant, a lubricant and a stabilizer that are conventional in the pharmaceutical field.

Use of the complex in preparation of drugs for treating psoriasis is provided.

### 3. Beneficial Effects

The present invention has the following advantages:

In the present invention, it is found for the first time that CD200 protein or CD200 fusion protein can treat psoriasis; it is found the present invention that CD200 protein and CD200 fusion protein can act on CD200R1 to alleviate imiquimod-induced psoriasis-like symptoms. CD200 protein and CD200 fusion protein inhibit the activation of inflammatory macrophages by inhibiting NF-T03 signal transduction, which leads to the termination of excessive proliferation of keratinocytes. After subcutaneous injection of CD200-Fc fusion protein during inflammation, the expression of macrophage-related pro-inflammatory factors IL-6, IL-1β and TNT-α is down-regulated, thereby achieving the effect of treating psoriasis.

Specifically, when administered in vitro, CD200 protein or CD200 fusion protein can inhibit the migration function of inflammatory macrophages, reduce the release of inflammatory factors, and inhibit the proliferation of keratinocytes, indicating that CD200 protein or CD200 fusion protein has therapeutic value. In vitro administration of a sufficient amount of CD200 protein and CD200 fusion protein is achieved by binding and activating CD200R1, thereby indicating that a CD200/CD200R1 pathway can treat psoriasis by inhibiting the activation of inflammatory macrophages, the release of inflammatory factors and the proliferation of keratinocytes.

In the present invention, the expression of CD200 in mice with psoriasis induced by IMQ began to decrease significantly from the second day after administration, the expression of CD200R1 also decreased significantly, and inflammatory factors increased significantly. After subcutaneous injection of CD200 fusion protein, the expression of CD200 and CD200R1 increased and the level of inflammatory factors decreased.

Although a lot of data shows that CD200 protein and CD200-Fc fusion protein play an important role in autoimmune diseases, their role in psoriasis is still unclear. In the present invention, it is found that CD200 protein and CD200-Fc fusion protein have the function of treating psoriasis, and novel use of the CD200 fusion protein in treating psoriasis is provided, providing a basis for developing its clinical application value.

### BRIEF DESCRIPTION OF DRAWINGS

FIGS. 1A and 1B are diagrams of a transwell chamber for culture of CD200 and macrophages, wherein FIG. 1A shows a control group, and FIG. 1B shows a treatment group with a CD200 protein or a CD200-Fc fusion protein;

FIGS. 2A-2D are diagrams showing the inhibition of macrophage migration in vitro by CD200 protein and CD200-Fc fusion protein, wherein FIG. 2A shows the function of CD200 protein, FIG. 2B shows the function of a CD200 IgG1 fusion protein, FIG. 2C shows the function of a CD200 IgG2 fusion protein, and FIG. 2D shows the function of a CD200 IgG3 fusion protein;

FIGS. 3A-3L show results of ELISA for inflammatory factors IL-1β, IL-6 and INF-α after treatment with CD200 protein and CD200-Fc fusion protein, wherein FIG. 3A-3C show the function of CD200 protein, FIG. 3D-3F show the function of CD200 IgG1 fusion protein, FIG. 3G-3I show the function of CD200 IgG2 fusion protein, and FIG. 3J-3L show the function of CD200 IgG3 fusion protein;

FIGS. 4A-4H show quantitative PCR results of an impact of CD200 protein and CD200-Fc fusion protein on the proliferation of keratinocytes, wherein FIG. 4A-4B show the function of CD200 protein. FIG. 4C-4D show the function of CD200 IgG1 fusion protein, FIG. 4E-4F show the function of CD200 IgG2 fusion protein, and FIG. 4G-4H show the function of CD200 IgG3 fusion protein;

FIGS. 5A-5C show NF-κB protein detection results after treatment with CD200-Fc fusion protein, wherein FIG. 5A shows a western blot result diagram, and FIG. 5B-5C are diagrams showing a fold change compared with a reference gene GAPDH;

FIGS. 6A and 6B are diagrams showing results of a model of imiquimod-induced mouse psoriasis, wherein FIG. 6A is a diagram showing changes in a skin state of psoriasis mice, and FIG. 6B is a graph showing changes in the body weight of the psoriasis mice;

FIGS. 7A-7D are diagrams of a PASI score of imiquimod-induced skin psoriasis in mice, wherein FIG. 7A shows an infiltration score, FIG. 7B shows a skin lesion area score. FIG. 7C shows an erythema score, and FIG. 7D shows a total score;

FIG. 8 shows HE staining results of imiquimod-induced skin psoriasis in mice;

FIGS. 9A-9D are diagrams of a PASI score after treatment with CD200-Fc fusion protein, wherein FIG. 9A shows an infiltration score. FIG. 9B shows a skin lesion area score, FIG. 9C shows an erythema score, and FIG. 9D shows a total score; and

FIGS. 10A-10C show results of ELISA for inflammatory factors IL-1β, IL-6 and TNF-α in mice with psoriasis after treatment with CD200-Fc fission protein, wherein FIG. 10A shows IL-1β, FIG. 10B shows IL-6, and FIG. 10C shows TNT-α.

### DETAILED DESCRIPTION

The following examples illustrate the present invention in detail: The examples are implemented on the premise of taking the present invention as the technical solution, and a detailed implementation solution and process are given, but the scope of the present invention is not limited to the following examples. The conditions and methods that are not indicated in the following embodiments are implemented conventionally.

### Example 1

Inhibition of Macrophage Migration by CD200 Protein and CD200-Fc Fusion Protein

Macrophages play an important role in the occurrence and progression of psoriasis, and CD200 protein and fusion protein can inhibit the migration of the macrophages. A specific method and results are as follows:

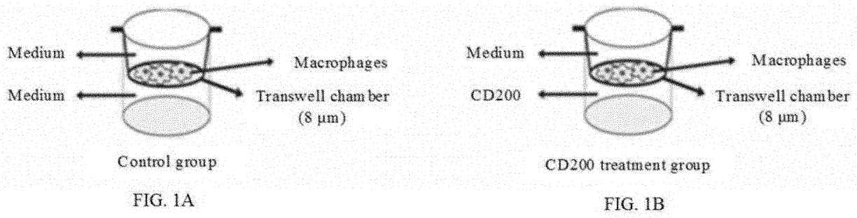

Method: Peritoneal macrophages needed to be obtained first. Mice were injected intraperitoneally with 1 nil of autoclaved 5% thioglycolate broth every day. After three consecutive days, the mice were killed, ascites of the mice was sucked out by using a syringe and washed with PBS, and the cells were cultured in DMEM containing penicillin (100 U/ml) streptomycin (100 mg/ml) and 10% fetal bovine serum (FBS). After 5 hours, the suspended cells were aspirated and washed three times with cold PBS to obtain adherent cells. The adherent macrophages were digested with 0.25% trypsin. The cell suspension in the DMEM medium was placed in an upper chamber of Matrigel-coated transwells (8 μm), and each insert contained 10⁵ cells. CD200 protein or CD200-Fc fusion protein was added into a lower chamber, as shown in FIGS. 1A and 1B. After incubation at 37° C. for 9 hours, the macrophages migrated to the lower chamber were counted.

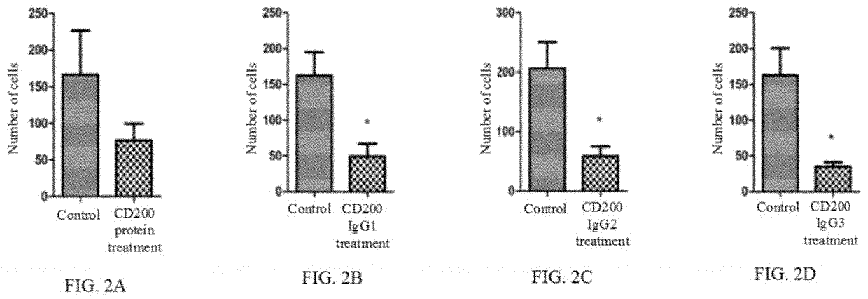

Results: As shown in FIGS. 2A-2D and Tables 1 to 4, transwell experiments showed that peritoneal macrophages in a control group could migrate to the other side of the transwell, while the migration of macrophages treated with CD200 protein and CD200-Fc fusion protein was significantly inhibited (P 0.05, Student's t-test).

TABLE 1
Effect of CD200 protein on macrophage migration

| | | Group | | | | | | |
|---|---|---|---|---|---|---|---|---|
| Item | | 1 | 2 | 3 | 4 | 5 | Mean ± SEM | P value |
| Number | control | 73 | 90 | 397 | 175 | 96 | 166.20 ± 60.319 | 0.202 |
| of cells | CD200 | 116 | 40 | 94 | 125 | 8 | 76.60 ± 22.631 | |
| | treatment | | | | | | | |

| | | | | | | | |
|---|---|---|---|---|---|---|---|
| Migration rate | 62.9% | 44.4% | 23.6% | 71.4% | 8.3% | 42.12 ± 11.792 | — |

TABLE 2
Effect of CD200 IgG1 fusion protein on macrophage migration

| | | Group | | | | | | |
|---|---|---|---|---|---|---|---|---|
| Item | | 1 | 2 | 3 | 4 | 5 | Mean ± SEM | P value |
| Number | control | 234 | 105 | 250 | 113 | 109 | 162.20 ± 32.701 | 0.016 |
| of cells | CD200 | 112 | 35 | 63 | 125 | 8 | 49.20 ± 17.825 | |
| | treatment | | | | | | | |

| | | | | | | | |
|---|---|---|---|---|---|---|---|
| Migration rate | 47.9% | 32.4% | 23.2% | 21.2% | 11.0% | 27.14 ± 13.874 | — |

TABLE 3
Effect of CD200 IgG2 fusion protein on macrophage migration

| | | Group | | | | | | |
|---|---|---|---|---|---|---|---|---|
| Item | | 1 | 2 | 3 | 4 | 5 | Mean ± SEM | P value |
| Number | control | 102 | 106 | 221 | 319 | 282 | 206.00 ± 44.489 | 0.014 |
| of cells | CD200 | 1 | 56 | 61 | 74 | 101 | 58.60 ± 16.379 | |
| | treatment | | | | | | | |

| | | | | | | | |
|---|---|---|---|---|---|---|---|
| Migration rate | 0.9% | 52.8% | 27.6% | 23.1% | 35.8% | 28.04 ± 18.937 | — |

TABLE 4
Effect of CD200 IgG3 fusion protein on macrophage migration

| | | Group | | | | | | |
|---|---|---|---|---|---|---|---|---|
| Item | | 1 | 2 | 3 | 4 | 5 | Mean ± SEM | P value |
| Number | control | 245 | 108 | 198 | 219 | 43 | 162.60 ± 37.755 | 0.013 |
| of cells | CD200 | 45 | 43 | 34 | 42 | 11 | 35.00 ± 6.285 | |
| | treatment | | | | | | | |

| | | | | | | | |
|---|---|---|---|---|---|---|---|
| Migration rate | 18.4% | 39.8% | 17.2% | 19.2% | 25.6% | 24.04 ± 9.392 | — |

### Example 2

Effect of Treatment with CD200 Protein and CD200-Fc Fusion Protein on Release of Inflammatory Factors

Inflammatory factors secreted by macrophages can aggravate the inflammatory response of psoriasis, while CD200 protein and fusion protein can inhibit the release of inflammatory factors from macrophages. A specific method and results are as follows:

Method: Peritoneum of normal mice and a model group were extracted and cultured. The process is as shown in Example 1. The cells were divided into three groups: a normal group, a treatment group with CD200 protein or CD200-Fc fusin protein, and a model group. After culture for 24 hours, the cell supernatant was collected for ELISA. A kit was purchased from Tianjin Anoric Biotechnology, and levels of IL-1β, IL-6 and TNF-α were detected.

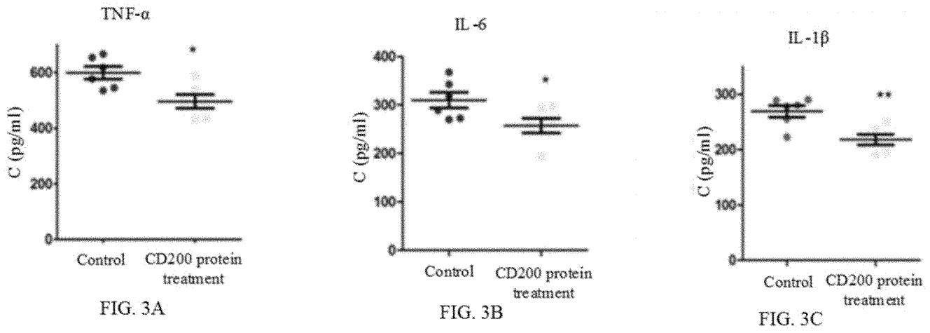

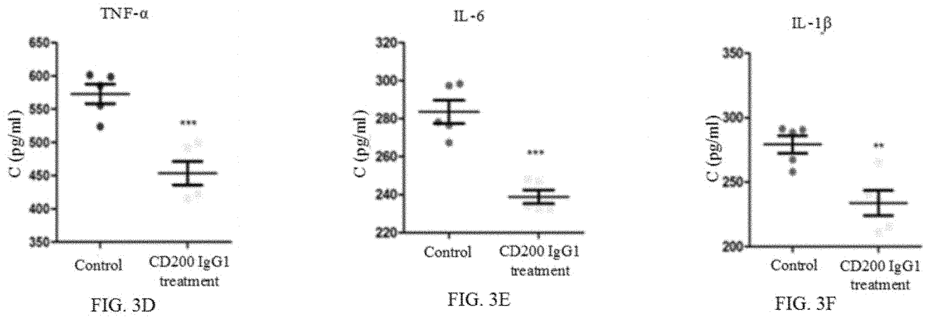

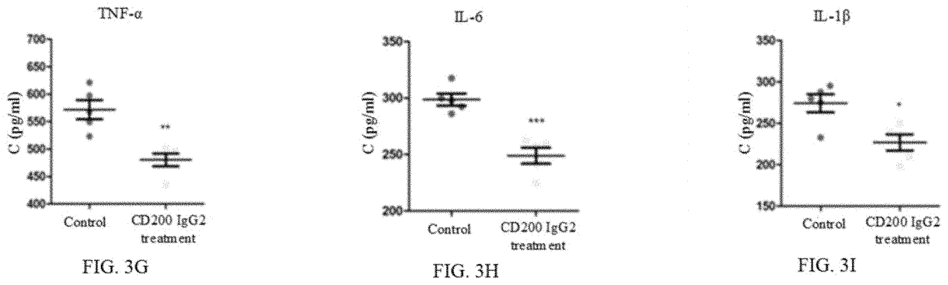

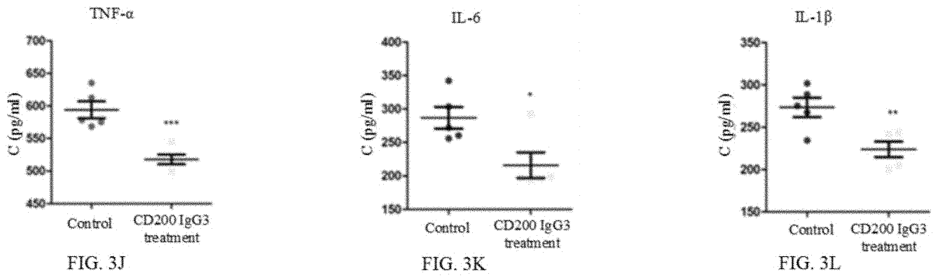

Results: After treatment with CD200 protein or CD200-Fc fusion protein, the levels of IL-1β (F (2, 12)=12.28, P=0.0012, Newman-Keuls' test), IL-6 (F (2, 12)=28.21. P<0.0001, Newman-Keuls' test) and TNF-α (F (2, 12)=17.09, P=0.0003, Newman-Keuls' test) were lower than those of the model group, as shown in FIGS. 3A-3L and Tables 5 to 8, indicating that CD200 protein and CD200-Fc fusion protein could reduce the release of inflammatory factors.

TABLE 5
Effect of CD200 protein on release of inflammatory factors

| | | Group | | | | | | |
|---|---|---|---|---|---|---|---|---|
| Item | | 1 | 2 | 3 | 4 | 5 | Mean ± SEM | P value |
| TNF-α | control | 545.4 | 577.4 | 653.4 | 617.4 | 535.4 | 585 80 ± 22.15 | 0.013 |
| | CD200 | 432.4 | 538.4 | 435.4 | 588.4 | 485.4 | 496.00 ± 30.14 | |
| | treatment | | | | | | | |
| IL-6 | control | 289.0 | 317.7 | 272.7 | 270.5 | 342.7 | 298.50 ± 13.93 | 0.121 |
| | CD200 | 246.5 | 297.0 | 194.0 | 259.0 | 292.7 | 258.00 ± 18.73 | |
| | treatment | | | | | | | |
| IL-1β | control | 222.8 | 255.3 | 289.0 | 290.3 | 277.8 | 267.00 ± 12.72 | 0.027 |
| | CD200 | 250.3 | 214.0 | 236.5 | 290.3 | 221.5 | 223.50 ± 9.42 | |
| | treatment | | | | | | | |

TABLE 6
Effect of CD200 IgG1 fusion protein on release of inflammatory factors

| | | Group | | | | | | |
|---|---|---|---|---|---|---|---|---|
| Item | | 1 | 2 | 3 | 4 | 5 | Mean ± SEM | P value |
| TNF-α | control | 585.2 | 523.7 | 598.4 | 601.3 | 555.4 | 574.80 ± 14.72 | 0.001 |
| | CD200 | 422.6 | 438.2 | 415.3 | 499.4 | 492.2 | 453.54 ± 17.68 | |
| | treatment | | | | | | | |
| IL-6 | control | 278.2 | 297.4 | 298.4 | 267.4 | 276.5 | 285.58 ± 6.12 | 0.001 |
| | CD200 | 232.5 | 247.0 | 234.0 | 248.2 | 232.6 | 238.86 ± 3.58 | |
| | treatment | | | | | | | |
| IL-1β | control | 290.5 | 288.5 | 258.0 | 291.3 | 267.4 | 279.14 ± 6.88 | 0.005 |
| | CD200 | 241.3 | 236.0 | 215.5 | 265.3 | 211.2 | 233.86 ± 9.74 | |
| | treatment | | | | | | | |

TABLE 7
Effect of CD200 IG2 fusion protein on release of inflammatory factors

| | | Group | | | | | | |
|---|---|---|---|---|---|---|---|---|
| Item | | 1 | 2 | 3 | 4 | 5 | Mean ± SEM | P value |
| TNF-α | control | 568.4 | 597.4 | 523.4 | 549.4 | 621.3 | 571.98 ± 17.26 | 0.003 |
| | CD200 | 482.4 | 501.5 | 435.7 | 496.5 | 485.4 | 480.30 ± 17.68 | |
| | treatment | | | | | | | |
| IL-6 | control | 286.0 | 297.7 | 292.3 | 299.5 | 317.5 | 298.60 ± 5.27 | 0.001 |
| | CD200 | 241.5 | 257.4 | 224.5 | 259.0 | 262.4 | 248.96 ± 7.09 | |
| | treatment | | | | | | | |
| IL-1β | control | 232.8 | 275.3 | 279.6 | 295.3 | 287.8 | 274.16 ± 0.89 | 0.002 |
| | CD200 | 198.3 | 209.0 | 236.4 | 250.3 | 239.4 | 226.80 ± 9.77 | |
| | treatment | | | | | | | |

TABLE 8
Effect of CD200 IgG3 fusion protein on release of inflammatory factors

| | | Group | | | | | | |
|---|---|---|---|---|---|---|---|---|
| Item | | 1 | 2 | 3 | 4 | 5 | Mean ± SEM | P value |
| TNF-α | control | 635.4 | 568.4 | 612.7 | 578.5 | 575.4 | 594.08 ± 12.85 | 0.002 |
| | CD200 | 499.5 | 513.4 | 515.4 | 516.0 | 545.2 | 517.90 ± 7.46 | |
| | treatment | | | | | | | |
| IL-6 | control | 256.0 | 303.5 | 272.0 | 260.5 | 342.2 | 286.84 ± 16.13 | 0.002 |
| | CD200 | 196.5 | 197.5 | 194.0 | 199.0 | 292.7 | 215.94 ± 19.21 | |
| | treatment | | | | | | | |
| IL-1β | control | 234.4 | 275.3 | 288.7 | 267.5 | 301.8 | 273.54 ± 17.40 | 0.0029 |
| | CD200 | 200.3 | 244.0 | 227.3 | 205.3 | 242.0 | 223.78 ± 9.07 | |
| | treatment | | | | | | | |

### Example 3

Effect of Macrophages on Keratinocyte Proliferation after Inhibition by CD200 Protein and CD200-Fc Fusion Protein

CD200 protein and CD200 fusion protein can indirectly inhibit the excessive proliferation of keratinocytes by inhibiting the activation of macrophages. Markers for keratinocyte proliferation used in this experiment were S100A7 and S100A8. A specific method and results are as follows:

Methods: Keratinocytes needed to be obtained first: Skin tissues were separated from neonatal mice and cut into pieces. The cut skin tissues were digested overnight with 25 U/ml neutral protease, and then digested with 0.05% trypsin-EDTA for 15 minutes. The cut skin tissues were washed with cold PBS, and suspended cells were incubated with a 154 CF medium. The keratinocytes were co-cultured with the macrophages treated with CD200 protein or CD200-Fc fusion protein in the remaining medium as the treatment group, and the keratinocytes were co-cultured with the untreated macrophages as the control group. After 48 hours, VCR detection was performed on the expression of S100A7 and S100A8 of keratinocytes. Primers used:

S100A7:
5′-GTACTCAGGTCATGGTTCTG-3′(upstream)
5′-GGTATTCAAGCAAGGTATCAC-3′(downstream)
S100A8:
5′-GGAGTTCCTTGCGATGGTGAT-3′(upstream)
5′-TCCTTGTGGCTGTCTTTGTGA-3′(downstream).

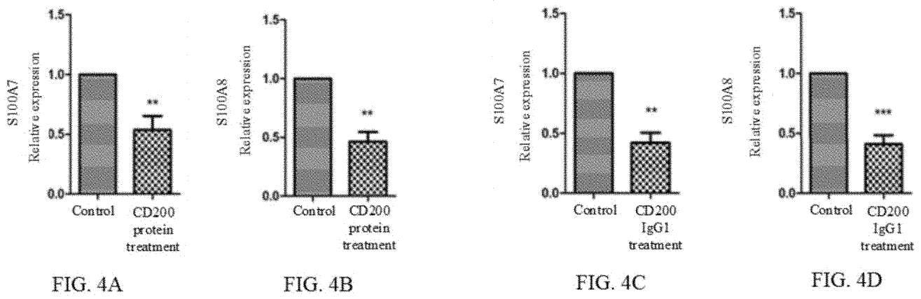

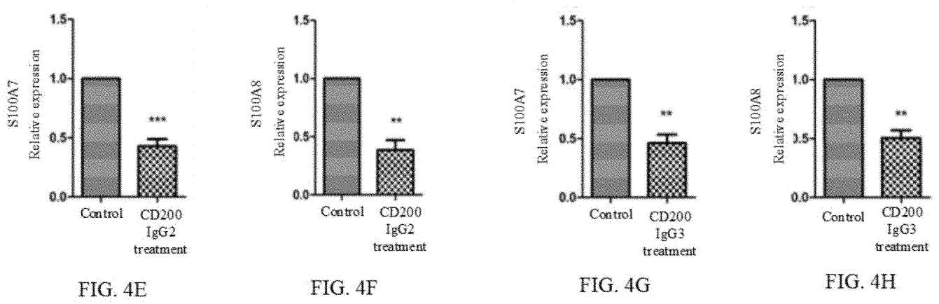

Results: As shown in FIGS. 4A-4H and Tables 9 to 12, it was found that the expression of S100A7 (P<0.01, Student's t-test) and S100A8 (P<1, Student's t-test) of keratinocytes after co-culture of keratinocytes and macrophages treated with CD200 protein and CD200-Fc fusion protein was significantly lower than that of the cells in the control group, indicating that CD200 protein and CD200-Fc fusion protein inhibited the proliferation of keratinocytes by inhibiting the activation of macrophages.

TABLE 9
Effect of CD200 protein on proliferation of keratinocytes

| | | Group | | | | | | |
|---|---|---|---|---|---|---|---|---|
| Item | | 1 | 2 | 3 | 4 | 5 | Mean ± SEM | P value |
| S100A7 | control | 1 | 1 | 1 | 1 | 1 | 1.00 ± 0.00 | 0.016 |
| | CD200 | 0.16 | 0.45 | 0.83 | 0.52 | 0.72 | 0.540 ± 0.116 | |
| | treatment | | | | | | | |
| S100A7 | control | 1 | 1 | 1 | 1 | 1 | 1.00 ± 0.00 | 0.003 |
| | CD200 | 0.44 | 0.32 | 0.58 | 0.71 | 0.27 | 0.46 ± 0.082 | |
| | treatment | | | | | | | |

TABLE 10
Effect of CD200 IgG1 fusion protein on proliferation of keratinocytes

| | | Group | | | | | | |
|---|---|---|---|---|---|---|---|---|
| Item | | 1 | 2 | 3 | 4 | 5 | Mean ± SEM | P value |
| S100A7 | control | 1 | 1 | 1 | 1 | 1 | 1.00 ± 0.00 | 0.002 |
| | CD200 | 0.34 | 0.23 | 0.54 | 0.68 | 0.32 | 0.42 ± 0.082 | |
| | treatment | | | | | | | |
| S100A7 | control | 1 | 1 | 1 | 1 | 1 | 1.00 ± 0.00 | 0.001 |
| | CD200 | 0.23 | 0.31 | 0.34 | 0.56 | 0.61 | 0.41 ± 0.074 | |
| | treatment | | | | | | | |

TABLE 11
Effect of CD200 IgG2 fusion protein on proliferation of keratinocytes

| | | Group | | | | | | |
|---|---|---|---|---|---|---|---|---|
| Item | | 1 | 2 | 3 | 4 | 5 | Mean ± SEM | P value |
| S100A7 | control | 1 | 1 | 1 | 1 | 1 | 1.00 ± 0.00 | 0.001 |
| | CD200 | 0.45 | 0.23 | 0.38 | 0.59 | 0.49 | 0.43 ± 0.060 | |
| | treatment | | | | | | | |
| S100A7 | control | 1 | 1 | 1 | 1 | 1 | 1.00 ± 0.00 | 0.002 |
| | CD200 | 0.67 | 0.16 | 0.30 | 0.42 | 0.38 | 0.39 ± 0.084) | |
| | treatment | | | | | | | |

TABLE 12
Effect of CD200 IgG3 fusion protein on proliferation of keratinocytes

| | | Group | | | | | | |
|---|---|---|---|---|---|---|---|---|
| Item | | 1 | 2 | 3 | 4 | 5 | Mean ± SEM | P value |
| S100A7 | control | 1 | 1 | 1 | 1 | 1 | 1.00 ± 0.00 | 0.002 |
| | CD200 | 0.71 | 0.32 | 0.41 | 0.54 | 0.32 | 0.48 ± 0.065 | |
| | treatment | | | | | | | |
| S100A7 | control | 1 | 1 | 1 | 1 | 1 | 1.00 ± 0.00 | 0.002 |
| | CD200 | 0.48 | 0.27 | 0.54 | 0.56 | 0.67 | 0.50 ± 0.066) | |
| | treatment | | | | | | | |

### Example 4

CD200-Fc Fusion Protein Inhibits Activation of Macrophages by Using an NT-κB Pathway.

CD200 protein and CD200 fusion protein can inhibit the activation of macrophages by using the NF-κB pathway, thereby indirectly inhibiting excessive proliferation of keratinocytes. A specific method and results are as follows:

Method: Some macrophages obtained from mouse ascites fell into two groups for culture, where CD200-Fc fusion protein was added into one group of medium as a CD200 treatment group, and one group using a normal medium was a control group. After culture for 48 hours, a half of the cells were taken for protein extraction, and the other half of the cells were taken for WB detection. Protein samples from the cells were isolated h SDS-PA E. and transferred to a poly vinylidene fluoride (PVDF) membrane. After being blocked in a 5% BSA TBST solution, the membrane was treated with a rabbit anti-mouse antibody specific for CD200RL GAPDH, NT-κB p50, and then HRP-bound goat anti-rabbit IgG was incubated as a secondary antibody. The membrane was washed and exposed to an X-ray film using an enhanced chemiluminescence reaction.

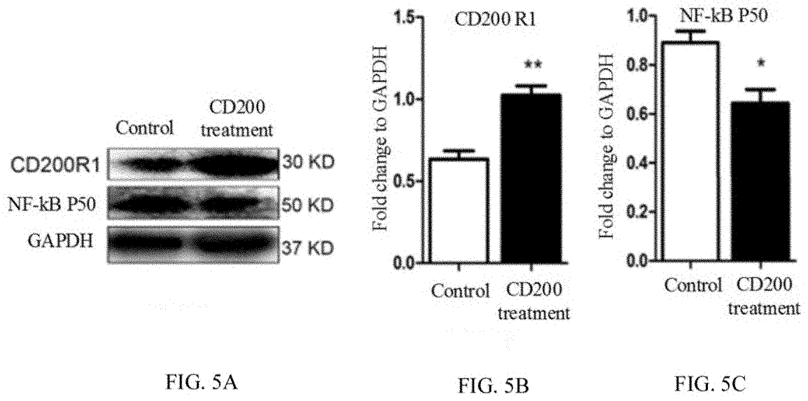

Results: As shown in FIGS. 5A-5C, compared with that of the cells in the control group, the protein expression of NF-κB p50 in the cells treated with CD200-Fc fusion protein was significantly reduced, indicating that CD200/CD200R1 is involved in reducing the expression of NF-κB p50 to inhibit macrophage activation.

### Example 5

Establishment and Verification of a Psoriasis Model on the Back of Mice Induced by Imiquimod (IMQ)

To verify therapeutic effect of CD200 protein and CD200-Fc fusion protein in mice, a mouse psoriasis model was established and evaluated. A specific method and results are as follows:

5.1 Establishment of a Psoriasis Model on the Back of Mice

Methods: Ten 5-6 week-old male BALB/c mice weighing 17-20 g were selected, including five as a control group and five as a model group. The back hair of the mice was removed, and a dose of 62.5 mg of a commercially available 5% INN cream was administered by application for 7 consecutive days, with vaseline as negative control. The skin state of the mice was recorded daily by photographing. The severity of back skin inflammation was measured by using PAST scoring criteria. PAST scores included erythema, scales, infiltration, and total score. The erythema, scales, and infiltration scores ranged from 0 to 4, which represented none, slight, mild, obvious, very obvious, respectively.

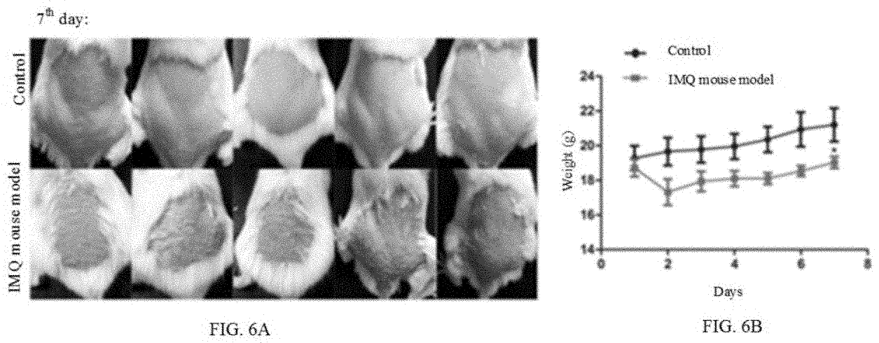

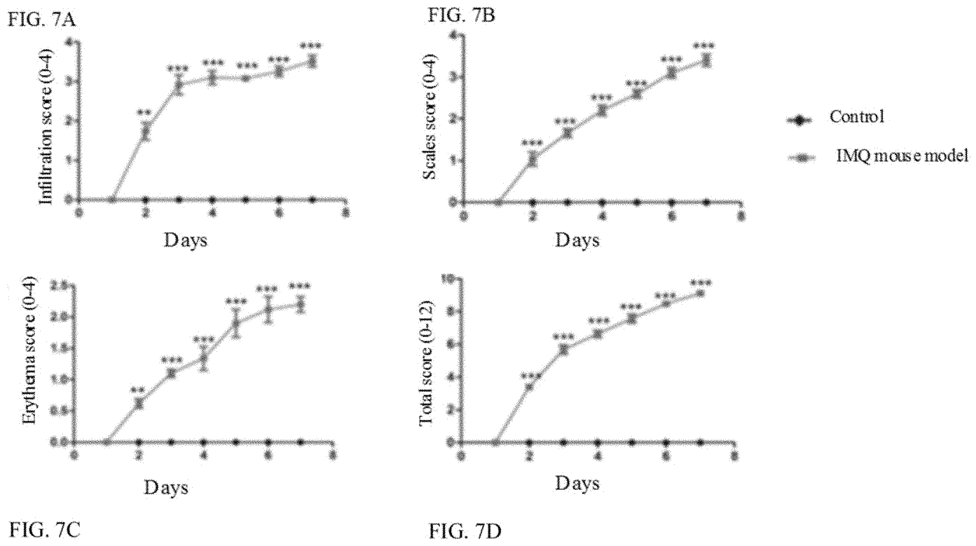

Results: Compared with mice of the control group, IMQ-stimulated mice lost weight from the second day, as shown in FIGS. 6A-6B. The severity of the skin injury was assessed by PAST scores and confirmed as deepening, as shown in FIGS. 7A-7D.

5.2 Validation of Skin Lesions of Psoriasis with HE Staining

Methods: The skin was taken out from the back of mice, fixed with 4% paraformaldehyde solution and embedded in paraffin. Paraffin-embedded (5-10 μm) sections were prepared and stained with Rh and examined under an optical microscope.

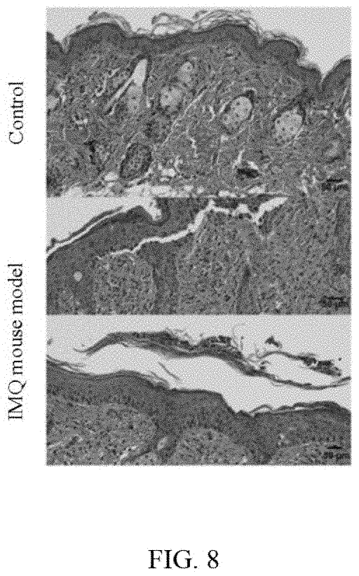

Results: As shown in FIG. 8, HE staining showed acanthosis, epidermal shedding, hyperkeratosis and cuticular layer thickening (P=0.01, Student's t-test). Based on the foregoing results, the appropriate time course, frequency and dose of IMQ stimulation were determined to successfully establish an IMQ-induced psoriasis-like mouse model.

### Example 6

Effect of CD200-Fc Fusion Protein on IMQ-Induced Psoriasis Symptoms

The effect of CD200 protein and CD200-Fc fusion protein on the in vivo treatment of psoriasis was evaluated. A specific method and results are as follows:

Methods: From the 3^{rd} day of IMQ-induced psoriasis inflammation, CD200 fusion protein was used for treatment (based on the results of the in vitro experiment, CD200 IgG2 Fc fission protein was selected as a experimental group for the in vivo experiment). CD200 fusion protein was injected once every two days by subcutaneous injection at the back. A dosage regimen is shown in Table 13.

TABLE 13
Therapeutic scheme for subcutaneous injection of CD200 fusion protein

| | Group | | |
|---|---|---|---|
| Item | control | CD200 treatment | IMQ-mouse-model |
| Model material | Vaseline | Imiquimod | Imiquimod |
| Type of | Normal saline | CD200-Fc | Normal saline |
| administration | | | |
| Administration | Subcutaneous | Subcutaneous | Subcutaneous |
| mode | injection | injection | injection |
| Administration | 200 μL | 200 μL (1 mg/kg) | 200 μL |
| dosage | | | |
| Administration | Once every | Once every | Once every |
| frequency | two days | two days | two days |
| Number in each | 5 | 5 | 5 |
| group | | | |

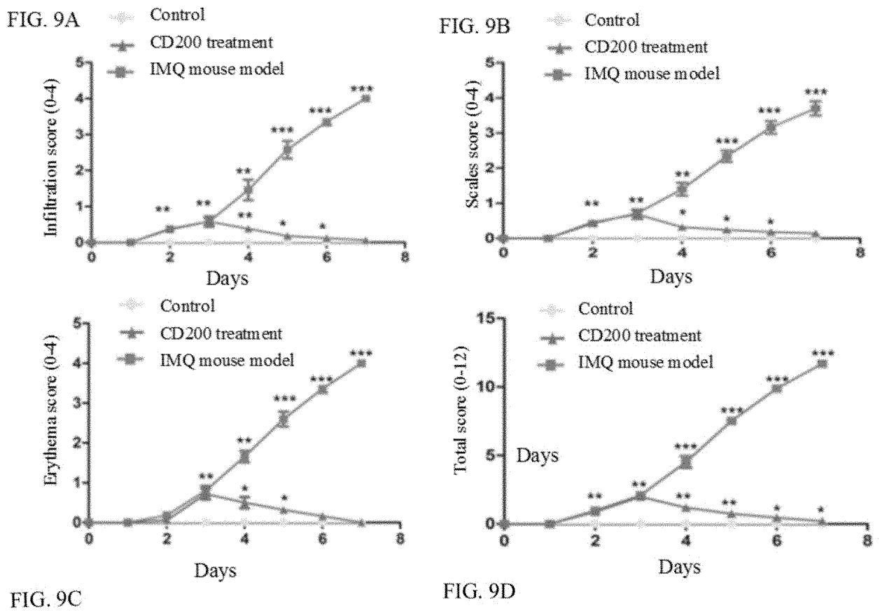

Results: As shown in FIGS. 9A-9D, compared with that of the model group, the skin lesions were significantly improved and the body weight loss was significantly reduced in the CD200 treatment group. In addition, PASI scores confirmed that infiltration, scales, and erythema were also diminished in mice in the CD200 treatment group.

### Example 7

Effect of CD200-Fc Fusion Protein on the Expression of Skin Inflammatory Factors

Method: The back skin of 200 mg mice was isolated and placed in a four-fold volume of PBS. After the skin was ground and centrifuged, the supernatant was collected for ELISA. A kit was purchased from Tianjin Anoric Biotechnology, and levels of IL-1β, TNF-α, and IL-6 were detected according to the specification.

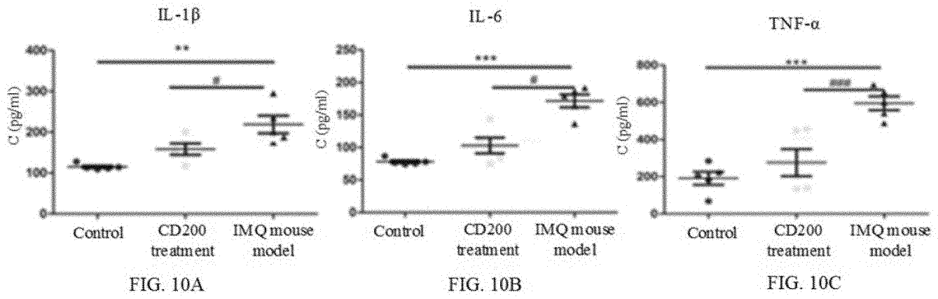

Results: As shown in FIGS. 10A-10C Table 14 and Table 15, the levels of IL-1β (F (2, 12)=12.28, P=0.0012. Newman-Keuls' test), IL-6 (F (2, 12)=28.21, P<0.0001. Newman-Keuls' test) and TNT-α (F (2, 123=17.09, P=0.0003, Newman-Keuls' test) were lower than those of the model group, indicating that CD200-Fc fusion protein could reduce the release of inflammatory factors.

TABLE 14
Effect of CD200 fusion protein on the expression of inflammatory factors of mouse skin

| | | Group | | | | | |
|---|---|---|---|---|---|---|---|
| Item | | 1 | 2 | 3 | 4 | 5 | Mean ± SEM |
| TNF-α | control | 202.4 | 284.4 | 220.4 | 181.4 | 68.4 | 187.20 ± 35.16 |
| | CD200 | 447.4 | 456.4 | 138.4 | 134.4 | 201.4 | 275.60 ± 72.96 |
| | treatment | | | | | | |
| | IMQ- | 487.4 | 694.4 | 540.4 | 653.4 | 559.4 | 584.00 ± 84.84 |
| | mouse- | | | | | | |
| | model | | | | | | |
| IL-6 | control | 86.50 | 74.00 | 75.25 | 77.75 | 76.50 | 78.0 ± 4.95 |
| | CD200 | 84.00 | 99.00 | 112.75 | 75.25 | 144.00 | 103.00 ± 27.03 |
| | treatment | | | | | | |
| | IMQ- | 186.5 | 179.0 | 136.5 | 164.0 | 191.5 | 171.50 ± 22.15 |
| | mouse- | | | | | | |
| | model | | | | | | |
| IL-1β | control | 109.00 | 111.50 | 112.75 | 114.00 | 127.75 | 115.00 ± 7.36 |
| | CD200 | 156.50 | 171.50 | 200.25 | 117.75 | 146.50 | 158.50 ± 30.49 |
| | treatment | | | | | | |
| | IMQ- | 295.25 | 187.75 | 175.25 | 200.25 | 235.25 | 218.75 ± 48.27 |
| | mouse- | | | | | | |
| | model | | | | | | |

1. Use of a CD200 extracellular domain protein or a fusion protein formed by a CD200 extracellular domain protein and an Fc fragment in preparation of drugs for treating psoriasis.

2. The use according to claim 1, wherein a nucleotide sequence of the CD200 extracellular domain protein is shown in SEQ ID NO: 1.

3. The use according to claim 1, wherein an amino acid sequence of the CD200 extracellular domain protein is shown in SEQ ID NO: 2.

4. The use according to claim 1, wherein Fc is an Fc fragment of human IgG1, IgG2 or IgG3, wherein a nucleotide sequence of the Fc fragment of the IgG1, IgG2 or IgG3 is shown in SEQ ID NO: 3, SEQ ID NO: 4 or SEQ ID NO: 5 respectively.

5. The use according to claim 4, wherein an amino acid sequence of the Fc fragment of the IgG1, IgG2 or IgG3 is shown in SEQ ID NO: 6, SEQ ID NO: 7 or SEQ ID NO: 8 respectively.

6. A complex, wherein the complex is obtained by adding one or more pharmaceutically acceptable excipients to the fusion protein according to claim 1.

7. The complex according to claim 6, wherein the excipients comprise a diluent, a filler, an adhesive, a wetting agent, an absorption enhancer, a surfactant, a lubricant and a stabilizer that are conventional in the pharmaceutical field.

8. Use of the complex according to claim 6 in preparation of drugs for treating psoriasis.


SEQUENCE LISTING (8 sequences)

SEQ ID NO: 1 - DNA, 696 bp, Artificial Sequence
  synthesized
atggagaggctggtgatcaggatgcccttctctcatctgtctacctacagcctggtttgggtcatggcagcagtggtgctgtgcacagcacaagtgcaagtggtgacccaggatgaaagagagcagctgtacacacctgcttccttaaaatgctctctgcaaaatgcccaggaagccctcattgtgacatggcagaaaaagaaagctgtaagcccagaaaacatggtcaccttcagcgagaaccatggggtggtgatccagcctgcctataaggacaagataaacattacccagctgggactccaaaactcaaccatcaccttctggaatatcaccctggaggatgaagggtgttacatgtgtctcttcaatacctttggttttgggaagatctcaggaacggcctgcctcaccgtctatgtacagcccatagtatcccttcactacaaattctctgaagaccacctaaatatcacttgctctgccactgcccgcccagcccccatggtcttctggaaggtccctcggtcagggattgaaaatagtacagtgactctgtctcacccaaatgggaccacgtctgttaccagcatcctccatatcaaagaccctaagaatcaggtggggaaggaggtgatctgccaggtgctgcacctggggactgtgaccgactttaagcaaaccgtcaacaaaggc

SEQ ID NO: 2 - protein, 150 aa, Artificial Sequence
  synthesized
MGSLVFRRPFCHLSTYSLIWGMAAVALSTAQVEVVTQDERKALHTTASLRCSLKTSQEPLIVTWQKKKAVSPENMVTYSKTHGVVIQPAYKDRINVTELGLWNSSITFWNTTLEDEGCYMCLFNTFGSQKVSGTACLTLYAHSTPSLQLF

SEQ ID NO: 3 - DNA, 1704 bp, Artificial Sequence
  synthesized
cctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggccggcaagcccccgctccccaggctctcggggtcgcgcgaggatgcttggcacgtaccccgtgtacatacttcccaggcacccagcatggaaataaa

SEQ ID NO: 4 - DNA, 525 bp, Artificial Sequence
  synthesized
atgctgcgaacagagagctgccgccccaggtcgcccgccggacaggtggccgcggcgtccccgctcctgctgctgctgctgctgctcgcctggtgcgcgggcgcctgccgaggtgctccaatattacctcaaggattacagcctgaacaacagctacagttgtggaatgagatagatgatacttgttcgtcttttctgtccattgattctcagcctcaggcatccaacgcactggaggagctttgctttatgattatgggaatgctaccaaagcctcaggaacaagatgaaaaagataatactaaaaggttcttatttcattattcgaagacacagaagttgggcaagtcaaatgttgtgtcgtcagttgtgcatccgttgctgcagctcgttcctcacctgcatgagagaagaatgaagagattcagagtggacgaagaattccaaagtccctttgcaagtcaaagtcgaggatattttttattcaggccacggaatggaagaaggtcagcagggttcatttaa

SEQ ID NO: 5 - DNA, 2685 bp, Artificial Sequence
  synthesized
atgacagcaatcaagcatgcattacaaagagacatttttacaccaaatgatgaacgcctgctgagcattgtgaatgtctgcaaagcaggaaaaaagaaaaagaactgttttttatgtgccacagtgacaactgaacgccctgtgcaggttaaggtggtcaaagtcaagaaatccgataagggagatttctacaaaaggcagattgcatgggcccttcgagatcttgctgtggtagatgccaaagatgctatcaaagaaaatcctgaatttgatttacactttgaaaaaatatataaatgggttgccagcagcactgctgaaaagaatgcatttatttcatgcatttggaaattgaatcagcgatatctccggaagaaaattgattttgtcaatgttagctcacagcttttggaagaatctgttccaagtggagaaaatcagagtgtgacaggaggtgatgaagaagtagtagatgaataccaagagttaaatgcaagagaagaacaggatatcgaaataatgatggaaggctgtgaatatgcaatctcgaatgcggaagcctttgcagaaaaattgtccagagagctgcaggtgctagatggggctaacatccagtcaatcatggcatctgaaaaacaagtcaacatcctgatgaaattgctagatgaggctctaaaggaggtagatcagattgaattgaaactgagcagttatgaggaaatgctccaaagtgtaaaagaacaaatggatcagatctctgaaagcaaccacctaattcatcttagtaacactaataatgtaaaactcctatctgagatagagttccttgtgaaccacatggacttggccaaaggtcatataaaggcccttcaggaaggagatcttgcttcttccagaggcattgaggcctgcaccaatgctgctgatgcccttctgcagtgcatgaatgtagctcttcgaccaggccatgacttgcttctggcagtcaaacagcaacagcagcgattcagtgatttgcgagagctttttgcccggagactggccagtcacctcaacaatgtttttgttcaacagggtcatgatcagagttcgactcttgcccaacactctgttgaactgactttacccaatcatcatccatttcatagagatttgctccgatatgccaagctgatggagtggctaaagagtacagattatggaaaatatgaaggactaacaaagaattacatggattatttatcccgactatatgaaagagaaatcaaagatttctttgaagttgcaaagatcaagatgactggcacaactaaagaaagcaagaagtttgctacactgcctcgaaaagaaagtgctgtcaaacaggaaacagagagtcttcatggaagttcggggaaattaactggatctacttctagtctaaataagctcagtgttcagagttcagggaatcgcagatctcagtcatcttccctgttggatatgggaaacatgtctgcctctgatctcgatgttgctgacaggaccaaatttgataagatctttgaacaggtactaagtgaactggagcccctatgtctggcagaacaggacttcataagtaaatttttcaaactacagcaacatcaaagtatgcctggaactatggctgaagcagaggacctggatggaggaacattatcacggcaacataattgtggcacaccactgcctgtttcatctgagaaagatatgatccgccaaatgatgattaaaatatttcgctgcattgagccagagctgaacaacctaattgcattaggagacaaaattgatagctttaactctctttatatgttagtcaaaatgagtcatcatgtgtggactgcacaaaatgtggaccctgcttctttcctaagtactacattgggaaatgttttggtgactgtcaaaaggaactttgacaaatgcattagtaaccaaataaggcaaatggaagaagtaaagatctcaaaaaagagtaaagttggaattcttccatttgttgctgaatttgaagaatttgctggacttgcagaatcaatcttcaaaaatgctgagcgtcgtggagacctggataaagcatacaccaaacttatcagaggagtatttgttaatgtggagaaagtagcaaatgaaagccagaagacccccagggatgtggttatgatggaaaactttcaccatatttttgcaactctttctcgattgaaaatctcatgtctagaagcagaaaaaaaagaagccaaacaaaaatacacagatcaccttcagtcttatgtcatttactctttaggacaacctcttgaaaaactaaatcatttctttgaaggtgttgaagctcgcgtggcacagggcataagggaggaggaagtaagttaccaacttgcatttaacaaacaagaacttcgtaaagtcattaaggagtaccctggaaaggaagtaaaaaaaggtctagataacctctacaagaaagttgataaacatttatgtgaagaagagaacttacttcaggtggtgtggcactccatgcaagatgaatttatacgccagtataagcactttgaaggtttgatagctcgctgttatcctggatctggtgttacaatggaattcactattcaggacattctggattattgttccagcattgcacagtcccactaa

SEQ ID NO: 6 - protein, 846 aa, Artificial Sequence
  synthesized
MLFTVSCSKMSSIVDRDDSSIFDGLVEEDDKDKAKRVSRNKSEKKRRDQFNVLIKELGSMLPGNARKMDKSTVLQKSIDFLRKHKEITAQSDASEIRQDWKPTFLSNEEFTQLMLEALDGFFLAIMTDGSIIYVSESVTSLLEHLPSDLVDQSIFNFIPEGEHSEVYKILSTHLLESDSLTPEYLKSKNQLEFCCHMLRGTIDPKEPSTYEYVKFIGNFKSLNSVSSSAHNGFEGTIQRTHRPSYEDRVCFVATVRLATPQFIKEMCTVEEPNEEFTSRHSLEWKFLFLDHRAPPIIGYLPFEVLGTSGYDYYHVDDLENLAKCHEHLMQYGKGKSCYYRFLTKGQQWIWLQTHYYITYHQWNSRPEFIVCTHTVVSYAEVRAERRRELGIEESLPETAADKSQDSGSDNRINTVSLKEALERFDHSPTPSASSRSSRKSSHTAVSDPSSTPTKIPTDTSTPPRQHLPAHEKMVQRRSSFSSQSINSQSVGSSLTQPVMSQATNLPIPQGMSQFQFSAQLGAMQHLKDQLEQRTRMIEANIHRQQEELRKIQEQLQMVHGQGLQMFLQQSNPGLNFGSVQLSSGNSSNIQQLAPINMQGQVVPTNQIQSGMNTGHIGTTQHMIQQQTLQSTSTQSQQNVLSGHSQQTSLPSQTQSTLTAPLYNTMVISQPAAGSMVQIPSSMPQNSTQSAAVTTFTQDRQIRFSQGQQLVTKLVTAPVACGAVMVPSTMLMGQVVTAYPTFATQQQQSQTLSVTQQQQQQSSQEQQLTSVQQPSQAQLTQPPQQFLQTSRLLHGNPSTQLILSAAFPLQQSTFPQSHHQQHQSQQQQQLSRHRTDSLPDPSKVQPQ

SEQ ID NO: 7 - protein, 174 aa, Artificial Sequence
  synthesized
MLRTESCRPRSPAGQVAAASPLLLLLLLLAWCAGACRGAPILPQGLQPEQQLQLWNEIDDTCSSFLSIDSQPQASNALEELCFMIMGMLPKPQEQDEKDNTKRFLFHYSKTQKLGKSNVVSSVVHPLLQLVPHLHERRMKRFRVDEEFQSPFASQSRGYFLFRPRNGRRSAGFI

SEQ ID NO: 8 - protein, 894 aa, Artificial Sequence
  synthesized
MTAIKHALQRDIFTPNDERLLSIVNVCKAGKKKKNCFLCATVTTERPVQVKVVKVKKSDKGDFYKRQIAWALRDLAVVDAKDAIKENPEFDLHFEKIYKWVASSTAEKNAFISCIWKLNQRYLRKKIDFVNVSSQLLEESVPSGENQSVTGGDEEVVDEYQELNAREEQDIEIMMEGCEYAISNAEAFAEKLSRELQVLDGANIQSIMASEKQVNILMKLLDEALKEVDQIELKLSSYEEMLQSVKEQMDQISESNHLIHLSNTNNVKLLSEIEFLVNHMDLAKGHIKALQEGDLASSRGIEACTNAADALLQCMNVALRPGHDLLLAVKQQQQRFSDLRELFARRLASHLNNVFVQQGHDQSSTLAQHSVELTLPNHHPFHRDLLRYAKLMEWLKSTDYGKYEGLTKNYMDYLSRLYEREIKDFFEVAKIKMTGTTKESKKFATLPRKESAVKQETESLHGSSGKLTGSTSSLNKLSVQSSGNRRSQSSSLLDMGNMSASDLDVADRTKFDKIFEQVLSELEPLCLAEQDFISKFFKLQQHQSMPGTMAEAEDLDGGTLSRQHNCGTPLPVSSEKDMIRQMMIKIFRCIEPELNNLIALGDKIDSFNSLYMLVKMSHHVWTAQNVDPASFLSTTLGNVLVTVKRNFDKCISNQIRQMEEVKISKKSKVGILPFVAEFEEFAGLAESIFKNAERRGDLDKAYTKLIRGVFVNVEKVANESQKTPRDVVMMENFHHIFATLSRLKISCLEAEKKEAKQKYTDHLQSYVIYSLGQPLEKLNHFFEGVEARVAQGIREEEVSYQLAFNKQELRKVIKEYPGKEVKKGLDNLYKKVDKHLCEEENLLQVVWHSMQDEFIRQYKHFEGLIARCYPGSGVTMEFTIQDILDYCSSIAQSH